(19) United States
(12) Patent Application Publication
(10) Pub. No.: US 2009/0152160 A1
(43) Pub. Date: Jun. 18, 2009

(54) Disposable diaper changing assembly

(75) Inventors: Keenan M. Thompson, Greenville, WI (US); Daniel J. Heuer, Lola, WI (US); Craig M. Thoms, Appleton, WI (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 12/002,034

(22) Filed: Dec. 14, 2007

(51) Int. Cl. B65D 69/00; A61B 19/00

(52) U.S. Cl. 206/581; 206/438

(57) ABSTRACT

A distinctive composite article has a package with a compartment for containing an article, and a drape. The drape has an end that is attached to an outer surface of the package. The drape is folded, and the package is folded about the drape for storage. In use, the package and then the drape are unfolded. The package may be opened to access the article(s) located within.

### BACKGROUND OF THE INVENTION

The present invention relates to a composite article for providing a desired combination of related and cooperating components for maintenance or cleaning operations. More particularly, the invention relates to a composite article for providing a related combination of components for the hygiene of an individual, such as the changing of a diaper on an infant.

Conventional systems for providing hygienic components for the care of an individual have included infant care bags for storing bottles, diapers, wet wipes, and other infant care supplies. Particular diaper changing bags have included closeable storage compartments, and have included carrying handles and foldable flaps.

Other systems have provided a package which included a disposable diaper, a separate changing pad and disposal container. The package could also include a closeable packet for retaining a wet wipe. Still other systems have included a disposable diaper with a diaper body having a pouch arranged to enclose a changing pad. The changing pad is removable from the pouch, and containers for holding personal care products, such as lotions, powders, oils, ointments, and wipes, are connected to the changing pad.

Conventional systems, such as those described above, have been cumbersome and unwieldy. Where components have been packaged together, it has been difficult to deploy the changing pad without scattering the other package contents. Where components such as changing pads and wet wipes are contained within the structure of a disposable diaper, the infant may be burdened with carrying about extra items which are bulky or uncomfortable. In other systems, a changing pad has been employed to package a diaper and possibly other related items. With the above systems, it has been difficult to simultaneously deploy the system while managing a squirming infant.

As a result, there has remained a need for a disposable assembly which conveniently and efficiently provides a desired combination of a drape or pad along with one or more other individual associated items for a given task. In particular, there has remained a need for a convenient and easy to use composite article which includes needed components for individual hygiene. For example, there has remained a need for a composite article which provides a changing pad and an individual disposable garment, along with optional wet wipe(s).

### SUMMARY OF THE INVENTION

Generally stated, the present invention provides a disposable assembly relating to the changing of a disposable garment. The assembly includes a drape defined by a sheet having a terminal end opposite a free end, and a drape width. The assembly further includes a package having a first compartment, a package width, and an accessible outer surface located opposite an attachment outer surface. The attachment outer surface defines an attachment area. There is a disposable garment disposed in the first compartment. The terminal end of the drape is attached to the attachment outer surface of the package so that only a portion of the attachment area is covered by the drape.

In another aspect of the present invention, a disposable assembly is used to protect a surface for performing a task. The disposable assembly includes a drape defined by a sheet having a terminal end opposite a free end. The assembly further includes a package having a first compartment and a second compartment, and an accessible outer surface opposite an attachment outer surface. The attachment outer surface defines an attachment area. A first article is disposed in the first compartment and a second article is disposed in the second compartment. The terminal end of the drape is attached to the attachment outer surface of the package so that only a portion of the attachment area is covered by the drape.

In yet another aspect of the invention is a method of protecting a surface for performing a change of an absorbent garment. The method includes a step of providing a disposable assembly in a storage condition, the disposable assembly comprising a drape defined by a sheet having a terminal end opposite a free end; a package having a first compartment; and a disposable garment disposed in the first compartment; wherein the terminal end of the drape is attached to the attachment outer surface of the package so that only a portion of the attachment area is covered by the drape. Other steps include: deploying the disposable assembly onto a support surface; opening the first compartment; and removing the absorbent garment.

When compared to conventional devices, such as those described above, the various aspects of the composite article of the invention can provide a more effective combination and presentation of associated and interrelated components. In particular, aspects the present invention can more efficiently provide a more readily usable and effective combination of personal hygiene components, such as a drape in the form of a changing pad, a disposable diaper, and a plurality of wet wipes. The composite article advantageously provides an ergonomically designed combination package which is more easily opened and manipulated. The drape can be easily unfolded while the other contained components are maintained in place. Once the drape is opened, the other components are conveniently presented for ready use. Advantageously, the assembly may be deployed with one hand.

### BRIEF DESCRIPTION OF THE DRAWINGS

The present invention will be more fully understood and further advantages will become apparent when reference is made to the following detailed description of the invention and the drawings, in which:

FIG. 1 shows a perspective view of one embodiment of a composite article of the present invention in a folded storage condition;

FIG. 1A shows a partial cross sectional view of the composite article of the present invention taken at line 1A-1A shown in FIG. 1;

FIG. 2 shows a perspective view of the composite article of FIG. 1, in an unused folded condition;

FIG. 3 shows a perspective view of the composite article shown in FIG. 1 in an unused deployed condition;

FIG. 4 shows a partial perspective view of the composite article of FIG. 3;

FIG. 5 shows a plan view of the composite article of FIG. 3, in a used deployed condition; and

FIG. 6 shows a plan view of one embodiment of an absorbent garment of the present invention.

### DETAILED DESCRIPTION OF THE INVENTION

The various embodiments of the invention will be described in the context of a disposable assembly 10 which includes a disposable absorbent garment article, such as a disposable diaper. Typically, the disposable articles are intended for limited use and are not intended to be laundered or otherwise cleaned for reuse. It is, however, readily apparent that the present invention could also be employed with other articles, such as children's training pants, adult incontinence products, feminine care products, items for bathing kits; items for dining; items for medical kits; items for shoe polish kits; items for use at the beach; and other applications where it is desirable to cover a surface with a drape while performing a particular task.

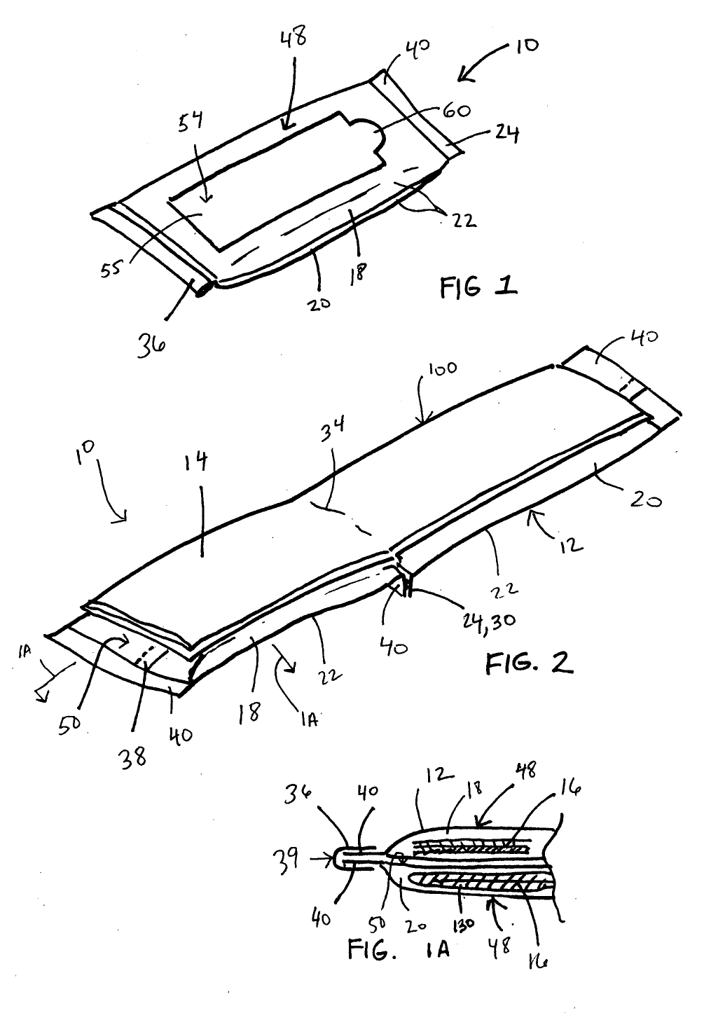

The disposable assembly 10 is has three main component categories, namely: a flexible package 12; a drape 14, which may be sized appropriately to function as a changing pad; and one or more articles 16, which may take many different forms, e.g. diapers and wipes. With reference to FIG. 3, a representative disposable assembly 10 is generally shown in its fully open and completely unfolded “deployed” condition. By contrast, the disposable assembly 10 of the invention can advantageously be folded and configured into the efficient, compact, storage condition shown in FIG. 1.

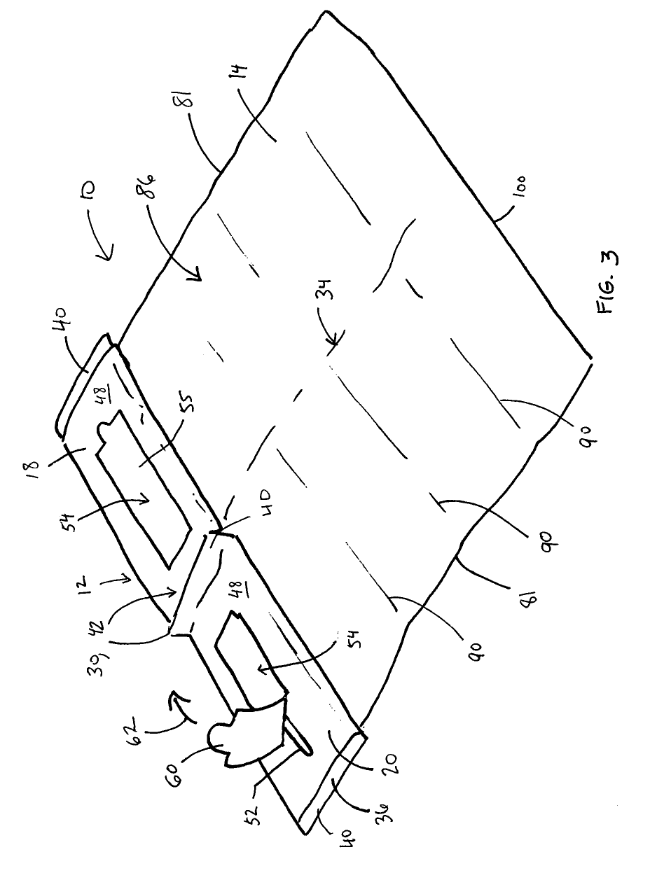

Package 12 includes one or more compartments for holding article(s) 16. In one exemplary embodiment shown in FIGS. 1 and 2, package 12 may be made from two separate packets 22 which are joined together at a joint 24. With reference to FIG. 2, each separate packet 22 defines a compartment, such as a first compartment 18 and a second compartment 20. However, more or less compartments are possible, as described herein.

In the various configurations of the invention, the package 12 is desirably composed of any operable sheet material, which is sufficiently flexible and a foldable, such as a woven fabric, a nonwoven fabric, a cellulosic sheet, a polymer film or the like, as well as combinations thereof. Suitable woven fabrics can include, for example, woven fabrics of cotton, rayon, linen, as well as other natural or synthetic fibers, and the like. Examples of suitable nonwoven fabrics include hydroentangled pulp, spunbond fabrics, SMS (spunbond-meltblown-spunbond) fabrics, SBL (stretch-bonded-laminate) fabrics, GORTEX fabrics, STL (stretch-thermal-laminate) fabrics or the like, as well as combinations thereof. Suitable polymer films include, for example, films composed of polyethylene, LDPE (low-density polyethylene), LLDPE (linear low-density polyethylene), ULDPE (ultra-low-density polyethylene), HDPE (high-density polyethylene), polypropylene, polyethylene/EVA (ethylene vinyl acetate) copolymers, a blend of polyethylenes, paper, nylon, cellophane, PVC (polyvinyl chloride) film, metallic foil, metalized films, polyester films, microporous breathable films or the like, as well as combinations thereof. In the representatively shown arrangement, the package 12 is a film composed of a low density polyethylene blend.

In particular aspects of the invention, the material of the package 12 is configured to be substantially liquid impermeable. For example, the package 12 can be constructed of a substantially liquid impermeable polymer film. Alternatively, the package 12 can be composed of a fabric which has been treated or otherwise configured to be substantially liquid impermeable. For example, the package 12 may be composed of a sheet comprising a laminate of a polymer film and a woven or nonwoven fabric layer.

Desirably, the material of the package 12 is configured to be thermally fusible. For example the package can include films or fibers which are heat-bondable. Accordingly, a bonding of the package can be accomplished by adhesive bonding, thermal bonding, thermal-mechanical embossing or crimping, ultrasonic bonding, or the like.

In the embodiment of the present invention shown in FIGS. 1-3, each packet 22 may be formed from a rectangular sheet of material (or a continuous sheet in an assembly line), folded together and bonded to form a flattened tube having a seam, such as the lap seam 38. In some applications, this folding step occurs after an article 16 has been introduced onto the sheet of material. Therefore, the act of folding causes the sheet to surround the article 16. At this stage of construction, the packet constructed from a single sheet has two opposite open ends, and the packet constructed from the continuous sheet has a series of spaced apart article 16 within a continuous tube. In the first instance, the open ends are bonded together to enclose the article 16 inside the packet. In the second instance, the tube is bonded together along the portions between the articles 16, and later cut into discrete units. Each single packet 22 ultimately has two opposite sealed ends 40. The finished packet further has an accessible surface 48, and an attachment surface 50 located oppositely thereof.

The packets may be placed in series such that two sealed ends 40 of the separate packets are aligned together and bonded to form a joint 42, such as the fin seam that is best seen in FIGS. 2 and 3. Other joints such as lap seams are contemplated.

While the package 12 is depicted as being constructed from two separate packets 22, other alternative constructions are possible. In one such alternative, package 12 may be a substantially unitary member composed of a single, unit sheet of tubular material. When the packets 22 are constructed from a continuous sheet or tubular material, they may remain integrally connected, and cut from the continuous tubular configuration two at a time. In yet another alternative, the package 12 is composed of a sheet material that is an assembly of a plurality of pieces joined and affixed along their edges to form a larger contiguous sheet.

Referring now to FIG. 3, in a particular aspect of the invention, the package 12 has at least one appointed fold line 30, which in the representative configuration, is shown extending generally along the longitudinal dimension of the deployed assembly, coinciding with the joint 42. Desirably, the fold line 30 has a location which corresponds to and substantially coincides with a location of at least one of the fold lines of the drape 14, such as longitudinally extending fold line 34. Accordingly, the package 12 and the drape 14 can be cooperatively folded about their respective, coinciding longitudinal fold lines 30, 34.

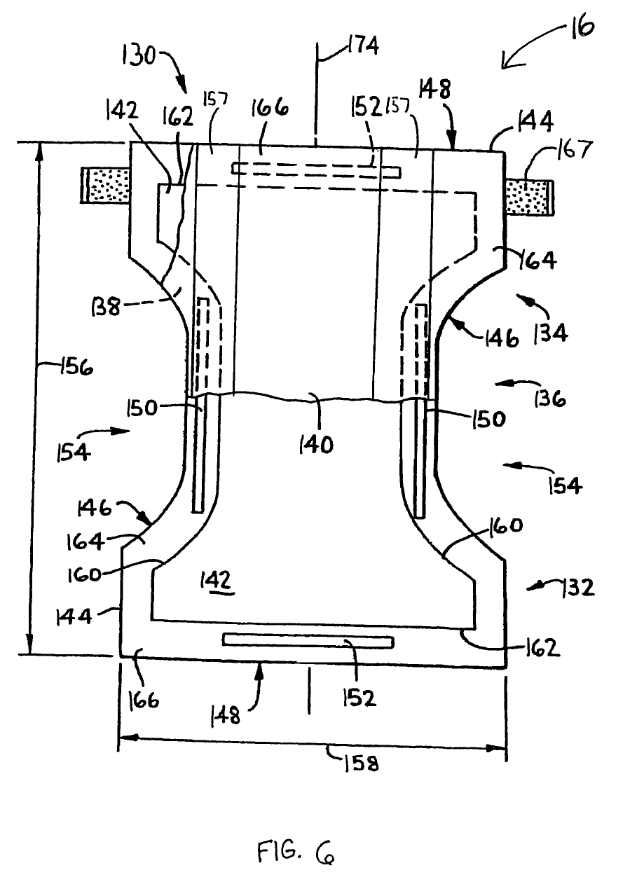

So that articles 16 may be readily accessed from each packet 22 without having to cut the packet or create an arbitrary tear thereon, an opening of some kind may be located on a surface of the packet. For instance, a finished packet 22 has length 44 and a width 46 (with respect to the longitudinal axis of the deployed or used disposable assembly 10, shown in FIG. 5). In the embodiment of the present invention shown in FIGS. 3 and 5, each packet 22 has an opening 52 is placed at its accessible surface 48. The opening may be in the form of a hole, such as the elongated hole shown, or in the alternative a series of perforations or a line of weakness that is broken by tugging at the packet 22, or by an opening mechanism as described below. Desirably, the opening 52 is placed on a surface in a location that will facilitate removal of article 16.

With reference to FIGS. 1 and 1A, a closure member 36 may be configured to hold the package 12 in a closed storage condition so the package 12 can contain and substantially envelop the drape 14 when the drape 14 is folded for the storage condition. Suitable closure mechanisms can, for example, include adhesive bonds, thermal bonds, sonic bonds, thermal-mechanical embossing, crimping, zippers, ZIPLOC® fasteners, hook-and-loop fasteners, stitching, pins, staples, clips, tapes, or the like, as well as combinations thereof. In the illustrated arrangement of FIGS. 1 and 1A, a closure 36 in the form of a tape serves to close the package 12 and provide the desired closed-package storage condition. In particular, as shown in FIG. 1A, the tape is folded lengthwise to cover the two aligned ends 40 to form a fold 39. The tape may be connected to the ends 40 with an adhesive or the like. The closure 36 may seal the entire length 44 of the ends 40, or may connect the ends 40 at a small percentage of the length 44.

In other aspects of the invention, the disposable assembly 10 may include an opening mechanism which allows a selective defeating of the closure 36 to provide a convenient access to the drape 14. For example, the article opening mechanism can include a system of frangible lines (not shown) which may be positioned along the folded edge 39 of the tape to selectively open the closure 36. Other possibilities include a string that is embedded in the tape and oriented along fold 39 such that when the string is pulled, the tape is separated at the fold 39. Examples of other releasable attachments include zippers, ZIPLOC® fasteners, hook-and-loop fasteners, releasable stitching or the like, as well as combinations thereof.

In other embodiments of the present invention, it is contemplated that various types of conventional packet configurations can be employed with the present invention. Examples of suitable packet designs and opening systems can include a packet with a ZIPLOC® opening or a hardpack with a latch opening, and the previously mentioned packet having a cover selectively closable with a resealable adhesive and a packet with a frangible seal. The type of opening may be determined by the contents inside the packet 22. For example, it may be desirable to close a packet containing wet wipes with a resealable adhesive cover. This will allow users to access a single wipe and reseal the package, should one be needed prior to deploying the package 12.

Thus, in particular configurations, the package 12 may includes an operative packet 22 opening system for selectively providing an access into the packet for removing the individual wipes for use, as desired. In one embodiment of the present invention, the opening system can include a removable panel 55 which can be selectively peeled away in a direction 62 to expose the opening 52 into the packet 22. The removable panel 55 may include a grasping pull-tab 60, and may or may not be completely detachable from the packet. A releasable seal can, for example, be provided by a releasable adhesive, or a releasable mechanical fastener, such as described elsewhere herein. However, if resealability is not desired, other adhesives or constructions may be used.

In an alternative embodiment of the present invention, the packet 22 opening system can include a line or other suitable region of frangibility, with or without a removable panel 55. It is further contemplated that the removable panel 55 may actually peel away with it a portion of the accessible surface 48 to simultaneously create opening 52. It is contemplated that there may be a single removable panel 55 covering any number of openings 52 located on the assembly surface 48. For example (not shown), a single removable panel could bridge the fold lone 30 and extend to cover both of the openings covered by the two removable panels 55 depicted in FIG. 3.

The disposable assembly 10 can further employ a displaying mechanism for presenting the composite article to user. For example, the displaying mechanism can comprise an aperture or hanger (neither shown) located on a sealed end 40 or a hanger located on the body of package 12, such as on a surface 48 or 50. The aperture or hanger allows a hanging of the composite article on a display hook or rod. The aperture may be located, and desirably centered, in a relatively shorter-length edge region of the package 12. Examples of other suitable displaying mechanisms can include hooks, tabs, straps, clips, pins, latches, adhesive strips or the like, as well as combinations thereof.

It should be readily appreciated that the disposable assembly 10 may include decorative and/or informational indicia and/or graphics on the package 12 (particularly on the accessible surface 48), the drape layer 14, the optional removable panel 55 (such as on outer surface 54), or any other component of the composite article. Any indicia visible from the outer surface of the package 12, including any indicia shown on a packet opening mechanism, may be oriented for intended display at any desired rotational angle of the disposable assembly 10. For example, the intended display of the indicia may be when the longer dimension of the closed package is aligned horizontally, or optionally may be when the shorter dimension of the closed package is aligned horizontally. Regardless, the indicia may be oriented so that it is most readily recognizable or readable when the product is displayed in the store.

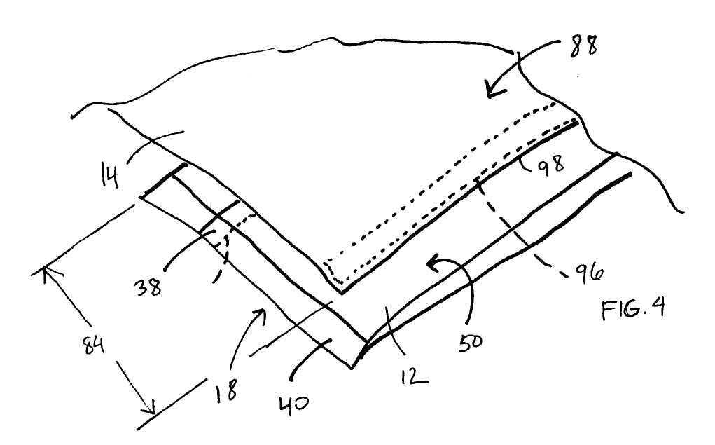

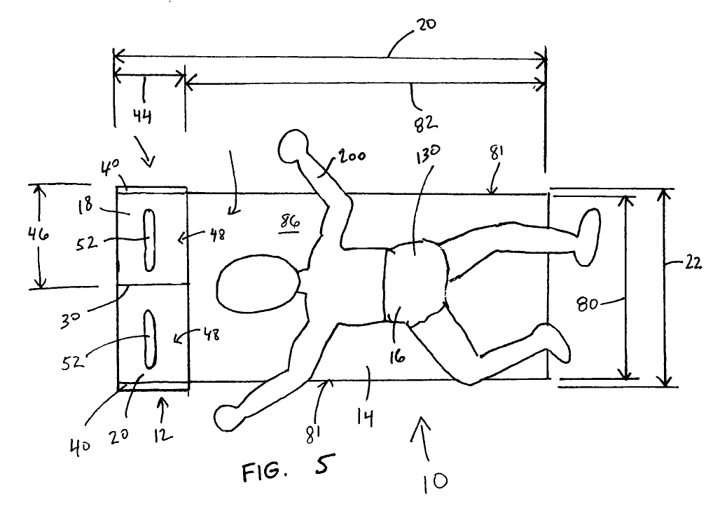

As illustrated in FIG. 1, the package 12 can form an effective envelope around the folded drape 14. Desired arrangements of the package 12 are dimensioned to form an efficient envelope about a folded drape 14. As seen in FIG. 5, the drape 14 has a drape width 80 that desirably, is less than the width 22 of the package 12 when it is fully spread out in a deployed condition. Width 80 is defined as the distance between drape side edges 81, FIGS. 3 and 5. The length 82 of drape 14 that is exposed is desirably long enough for the task at hand. For example, is the drape 14 operates as a changing pad, then it is desirably long enough for the size of baby corresponding to the diaper article 16 to effectively be protected from the table on which the drape 14 is spread out. As seen in FIG. 4, the drape 14 may have an additional length or “attachment length” 84 that is taken up in the attachment of the drape 14 to package 12, as described herein. The total length of the drape 14 is defined as the sum of the attachment length and the length 82.

The drape 14 has an appointed working surface 86 and an oppositely positioned support contact surface 88. Desirably, the working surface 86 is appointed to be a user side surface of the drape 14. The support contact surface 88 is appointed to be placed against the ground, floor, table, bed, or whatever surface is used to support the activity associated with the disposable assembly 10, such as the diaper changing example shown in the figures.

The drape 14 may also be configured to exhibit an increased coefficient of friction to help reduce slippage or other movement of the disposable assembly 10 relative to its underlying support surface, or to prevent slippage of whatever object is placed on the working surface 86 of drape 14. Various types of techniques may be employed to adjust the frictional coefficient. For example, the coefficient of friction can be increased by embossing the surfaces 86 and/or 88 of the drape 14, or by applying a high-friction surface treatment, applying adhesives, employing blooming slip agents, coatings, applying chemical/electrical discharge treatments, or the like to such surfaces.

In the various configurations of the invention, the drape 14 may be composed of any operative sheet material which is sufficiently flexible and foldable, such as a woven fabric, a nonwoven fabric, a paper layer, a polymer film or the like, as well as combinations thereof. The drape 14 may be a substantially unitary member composed of a single, unit sheet of material, or may be an assembly composed of a plurality of pieces joined and affixed along their edges to form a larger contiguous sheet. Suitable woven fabrics include, for example, fabrics of cotton, rayon, linen, as well as other natural or synthetic fibers, and the like. Suitable nonwoven fabrics include hydroentangled pulp, spunbond fabrics, SMS (spunbond-meltblown-spunbond) fabrics, SBL (stretch-bonded-laminate) fabrics, GORTEX® fabrics, STL (stretch-thermal-laminate) fabrics or the like, as well as combinations thereof. Suitable polymer films include films composed of polyethylene, LDPE (low-density polyethylene), LLDPE (linear low-density polyethylene), ULDPE (ultra-low-density polyethylene), HDPE (high-density polyethylene), polypropylene, polyethylene/EVA (ethylene vinyl acetate) copolymers, a blend of polyethylenes, paper, nylon, cellophane, PVC (polyvinyl chloride) film, metallic foil, metalized films, polyester films, microporous, breathable films or the like, as well as combinations thereof.

For example, the drape 14 may include a nonwoven fabric layer portion laminated to a barrier layer portion. The fabric portion is suitably joined and secured to the barrier portion with a suitable attachment mechanism, such as adhesive bonding, thermal bonding, sonic bonding, stitching, pinning, stapling, clipping, entangling, another system of chemical or mechanical interaction or the like, as well as combinations thereof. In one non-limiting yet desired configuration, the drape 14 includes a nonwoven fabric portion at the working surface 86, the fabric portion composed of a polypropylene spunbond fabric provided at a basis weight of about 10 to about 40 gsm. The fabric portion is laminated to a barrier film portion located at the support contact surface 88, which has a total film thickness of about 0.5 to about 5 mils and may be formed from polyethylene.

In particular aspects of the invention, the drape 14 can be configured to be substantially liquid impermeable. For example, the drape 14 can include a substantially liquid impermeable polymer film. In other arrangements, the drape 14 can include a woven or nonwoven fabric which is water repellent, or has been treated or otherwise configured to be substantially liquid impermeable. For example, a woven or nonwoven fabric can be coated or otherwise treated with a water repellent material to impart an operative level of liquid impermeability. In a desired configuration, the drape 14 can be a laminate member composed of a nonwoven, spunbond polypropylene fibrous layer and a polypropylene catalloy barrier film. The working surface may also be treated with anti-bacterial and/or anti-viral agent.

In particular other aspects of the invention, the drape 14 can be configured to be insulated. For example, the drape 14 may include a closed-cell foam layer to provide a measure of insulation. This may be advantageous when placing a baby on a drape supported by a cold surface.

Various types of articles 16 may be incorporated into the disposable assembly 10 of the present invention. For example, the article 16 can include a child's diaper or training pant, an adult incontinence product, a feminine care product, an item for shining shoes, an item for jewelry cleaning, an item for medical treatment, an item for first-aid, an item for dining, an item items for use at the beach, or the like.

In the representatively shown arrangements, a first article 16 can comprise a disposable absorbent article, such as the disposable diaper 130 shown in FIG. 5. Desirably, there is only one diaper 130 included in each assembly 10. However, it is contemplated that there may be two or three diapers included in an assembly 10. The disposable diaper 130 can have any diaper configuration, with one non-limiting example described below. In the non-limiting example shown in FIG. 1A, the diaper is located in the second compartment 20.

In other embodiments of the present invention, the composite article 16 can include take the form of a toy, diaper powder and/or lotion, a disposal bag, or the like, as well as combinations thereof. The packet may contain one or more wet wipes or towelettes. In the non-limiting example shown in FIG. 1A, the two wet wipes are located in the first compartment 18. The individual wipes may also have conventional configurations. For example, wet wipes may be constructed from a any web suitable for use as a wet wipe, including meltblown, coform, airlaid, bonded-carded web, and the like and can comprise synthetic or natural fibers or combinations thereof. One non-limiting example of web for a wipe basesheet is a coform basesheet of polypropylene and cellulose fibers having a basis weight of about 70 grams per square meter and manufactured generally as described in U.S. Pat. No. 4,100,324 to Anderson et al. dated Jul. 11, 1978, which is herein incorporated by reference. The basesheet is then saturated or otherwise impregnated with the wet wipe solution by any suitable means such as spraying, dipping, or the like. Add-on levels of solution can range from about 100 to about 700 weight percent, based on the dry weight of the basesheet. The desired amount will depend greatly on the nature of the basesheet.

At least a portion of the flexible drape 14 is affixed to the package 12, and more particularly, the drape 14 is at least partially overlapped or otherwise superposed on the package 12. In the shown arrangement, the drape 14 is superposed over a portion of the entire attachment area defined by the attachment surface 50 of package 12. Desirably, about 5 to about 95 percent of the attachment area is covered by the drape. In the alternative, about 10 to about 85 percent of the attachment area is covered by the drape. In yet another alternative, about 20 to 60 percent of the attachment area is covered by the drape 14.

In particular, to attach drape 14 to package 12, the working surface 86 may be positioned immediately adjacent the attachment surface 50 of the package, as shown in FIGS. 3 and 4. A bond may be formed between the surface 50 and the working surface of drape 14 using an adhesive or the like. For example, as shown in FIG. 4, a continuous or discontinuous layer of adhesive 96 may be disposed near a terminal edge 98 of the drape 14. (Terminal edge 98 is opposite the free edge 100, shown in FIGS. 2 and 3.)

In the alternative, the package 12 can be pivotably attached to the support contact surface 88 of the drape 14 at a location which is generally adjacent to the terminal edge 98 of drape 14. In this arrangement, the package 12 is pivotably movable to the deployed condition, as determined when the drape 14 is in its unfolded condition and disposed on a support with the working surface 86 facing the user.

In an optional aspect of the invention, the package 12 can be releasably attached to the drape 14. The releasability can, for example, be provided by a frangible bond between the package 12 and the drape 14. The frangible bond can, for example, be provided by an appointed region of frangibility, a releasable adhesive fastener, a releasable mechanical fastener, or the like. Examples of releasable mechanical fasteners include snaps, zippers, ZIPLOC fasteners, cooperating hook-and-loop fasteners (including, e.g. mushroom-and-loop fasteners), clips, other cooperating systems of interengaging mechanical elements, or the like, as well as combinations thereof.

With reference to FIGS. 3 and 5, the representatively shown configuration of the drape 14 includes longitudinal fold line 34, which effectively delimits or divides the open drape width 80 into two laterally adjacent, segments or sections. One or more lateral fold lines 90 can provide for two or more longitudinally adjacent segments. It should be readily apparent that the additional longitudinal fold lines may optionally be incorporated to divide the total drape width into further segments. In the various arrangements, the resultant width-wise and/or longwise segments of the drape layer can be unequal or substantially equal in width, as desired.

In the shown configurations, the location of the longitudinal fold line 34 generally corresponds to the location of the package fold line 30. Desirably, the drape 14 may be substantially centered with respect to the width of the package 12 (e.g. FIG. 5), or may be asymmetrically offset towards one side of the package, when the drape is in its fully opened, unfolded condition (not shown).

The folds with respect to the lateral fold lines 90 may all be arranged to turn or pivot in generally the same direction, or may be arranged to pivot in a zigzag, accordion-type configuration. In addition, the drape 14 may be folded along its lateral fold lines in any suitable sequence.

In operation, the package 12 is unfolded so that the drape 14 can be unfolded to its open, deployed position. The first compartment 18 and second compartment 20, such as the diaper and the wipes compartments, can then be readily accessed and used. The unfolding of the drape 14 and the movement or removal of the contained unit components can occur in a sequence which is substantially a reverse of the sequence which was employed to enclose the unit components, and to fold the drape 14 and package 12.

In another embodiment of the present invention, there is a method of protecting a surface for performing a change of an absorbent garment, such as a diaper 130. Provided is the disposable assembly 10 in a folded, storage condition that is desirably fastened with a closure 36. The assembly 10 is then deployed. Deployment may occur by opening the closure 36; unfolding the package 12 to reveal the folded drape 14, unfolding the drape 14; and placing the support contact surface of the drape onto a support surface. The drape 14 may be grasped at the free end 100 of the drape 14 and spread out on the support surface such as a floor or table. Desirably, the working surface 86 faces the user, and the opposite surface 88 makes contact with the support. A baby 200 may be disposed on the surface 86 in any position the user chooses. The compartments 18, 20 may be opened as desired to remove the articles 16 stored within. For example, the compartment 18 may be opened to obtain a wet wipe, and the compartment 20 may be opened to obtain the diaper 130. The compartments may be opened by breaking a frangible seal or removing a removable panel as described above.

### Exemplary Absorbent Garment

FIG. 6 illustrates a disposable absorbent article, and more specifically a diaper 130. Examples of other suitable disposable absorbent articles that can be used with the disposable assembly 10 of the present invention include, but are not limited to, disposable absorbent pants, training pants, feminine care products, incontinence products, disposable apparel, or the like.

The diaper 130 is shown in FIG. 6 in an unfolded, flat-out, uncontracted state (i.e., with all elastic induced gathering and contraction removed). Portions of the structure are partially cut away to more clearly show the interior construction of the diaper 130, with the surface of the diaper 130 which contacts the wearer facing the viewer. FIG. 6 illustrates a disposable diaper 130 as having a front region 132, a rear region 134 and a crotch region 136 located between the front and rear regions. The diaper 130 comprises a backsheet 138, a topsheet 140, and an absorbent core 142 situated between the backsheet and the topsheet. The outer edges of the diaper 130 define a periphery 144 with transversely opposed, longitudinally extending side edges 146; longitudinally opposed, transversely extending end edges 148; and a system of elastomeric gathering members, such as a system including leg elastics 150 and waist elastics 152. The longitudinal side edges 146 define the leg openings 154 for the diaper 130, and optionally, are curvilinear and contoured. The transverse end edges 148 are illustrated as straight, but optionally, may be curvilinear. The diaper 130 may also comprise additional components to assist in the acquisition, distribution and storage of bodily waste. For example, the diaper 130 may comprise a transport layer, such as described in U.S. Pat. No. 4,798,603, issued to Meyer et al., or a surge management layer, such as described in European Patent Application Publication No. 0 539 703, published May 5, 1993.

The diaper 130 generally defines a longitudinally extending length dimension 156, and a laterally extending width dimension 158, as representatively illustrated in FIG. 6. The diaper 130 may have any desired shape, such as rectangular, I-shaped, a generally hourglass shape, or a T-shape.

The backsheet 138 defines a length and a width that, in the illustrated version, coincide with the length and width of the diaper 130. The absorbent core 142 generally defines a length and width that are less than the length and width of the backsheet 138, respectively. Thus, marginal portions of the diaper 130, such as marginal sections of the backsheet 138, may extend past the transversely opposed, longitudinally extending terminal side edges 160 and/or the longitudinally opposed, transversely extending terminal end edges 162 of the absorbent core 142 to form side margins 164 and end margins 166 of the diaper 130. The topsheet 140 is generally coextensive with the backsheet 138, but may optionally cover an area that is larger or smaller than the area of the backsheet, as desired. The backsheet 138 and topsheet 140 are intended to face the garment and body of the wearer, respectively, while in use. The topsheet 140 and the backsheet 138 can, for example, be joined to each other in at least a portion of the diaper periphery 144 by attachment mechanisms (not shown) such as adhesive bonds, sonic bonds, thermal bonds, pinning, stitching, or a variety of other attachment techniques known in the art, as well as combinations thereof.

The topsheet 140 suitably presents a bodyfacing surface which is compliant, soft feeling, and non-irritating to the wearer's skin. Further, the topsheet 140 may be less hydrophilic than the absorbent core 142, to present a relatively dry surface to the wearer, and is sufficiently porous to be liquid permeable, permitting liquid to penetrate readily through its thickness. A suitable topsheet 140 may be manufactured from a wide selection of web materials, such as porous foams, reticulated foams, apertured plastic films, natural fibers, synthetic fibers (for example, polyester or polypropylene fibers), or a combination of natural and synthetic fibers. The topsheet 140 is suitably employed to help isolate the wearer's skin from liquids held in the absorbent core 142.

Various woven and nonwoven fabrics may be used for the topsheet 140. For example, the topsheet 140 may be composed of a meltblown or spunbonded web of polyolefin fibers. The topsheet 140 may also be a bonded-carded web composed of natural and/or synthetic fibers. The topsheet 140 may be composed of a substantially hydrophobic material, and the hydrophobic material may, optionally, be treated with a surfactant, or otherwise processed, to impart a desired level of wettability and hydrophilicity. Specifically, the topsheet 140 may be a nonwoven, spunbond, polypropylene fabric.

The backsheet 138 may suitably be composed of a material which is either liquid permeable or liquid impermeable. It is generally desirable that the backsheet 138 be formed from a substantially liquid impermeable material. For example, a typical backsheet 138 can be manufactured from a thin plastic film or other flexible liquid impermeable material. Further, the backsheet 138 may be formed of a woven or nonwoven fibrous web layer which has been totally or partially constructed or treated to impart a desired level of liquid impermeability to selected regions that are adjacent or proximate the absorbent core 142. Still further, the backsheet 138 may optionally be composed of micro-porous “breathable” material that permits vapors to escape from the absorbent core 142 while still preventing liquid exudates from passing through the backsheet.

The absorbent core 142 may comprise a matrix of hydrophilic fibers, such as a web of cellulosic fluff, mixed with particles of a high-absorbency material commonly known as superabsorbent material. In a particular version, the absorbent core 142 comprises a mixture of superabsorbent hydrogel-forming particles and wood pulp fluff. The wood pulp fluff may be exchanged with synthetic polymeric, meltblown fibers or with a combination of meltblown fibers and natural fibers. The superabsorbent particles may be substantially homogeneously mixed with the hydrophilic fibers or may be non-uniformly mixed.

The high-absorbency material can be selected from natural, synthetic and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble, but swellable.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful. Processes for preparing synthetic, absorbent gelling polymers are disclosed in U.S. Pat. No. 4,076,663, issued to Masuda et al., and U.S. Pat. No. 4,286,082, issued to Tsubakimoto et al.

As representatively illustrated in FIG. 6, the diaper 130 may include a pair of containment flaps 157 that are configured to provide a barrier to the lateral flow of body exudates. The containment flaps 157 may be located along the longitudinally extending side edges 146 of the diaper 130 adjacent the side edges of the absorbent core 142. Each containment flap 157 typically defines an unattached edge that is configured to maintain an upright, perpendicular configuration in at least the crotch region 136 of the diaper 130 to form a seal against the wearer's body. The containment flaps 157 may extend longitudinally along the entire length of the absorbent core 142 or may only extend partially along the length of the absorbent core 142. When the containment flaps 157 are shorter in length than the absorbent core 142, the containment flaps 157 can be selectively positioned anywhere along the side edges 146 of the diaper 130 in the crotch region 136. The containment flaps 157 may extend along the entire length of the absorbent core 142 to better contain the body exudates.

The diaper 130 may further include elastics at the end edges 148 and side edges 146 of the diaper 130 to further prevent leakage of body exudates and support the absorbent core 142. The diaper 130 may also include a pair of waist elastics 152 that are connected to the end edges 148 of the diaper 130. The leg elastics 150 and waist elastics 152 are generally adapted to fit about the legs and waist of a wearer in use to maintain a positive, contacting relationship with the wearer to effectively reduce or eliminate the leakage of body exudates from the diaper 130.

The elastics may be adhered to the backsheet 138 in a stretched position, or they may be attached to the backsheet 138 while the backsheet 138 is pleated, such that elastic constrictive forces are imparted to the backsheet 138. The leg elastics 150 may also include such materials as polyurethane, synthetic and natural rubber. The waist elastics 152 may be formed by elastic strands attached to the backsheet 138 or they may be formed by attaching separate pieces of stretchable materials to the waist regions of the article.

The disposable absorbent articles can but need not necessarily comprise fasteners 167 for securing the absorbent article about the waist of the wearer. The illustrated versions of the diaper 130 comprise such fasteners 167. In at least one version, the fasteners 167 are situated in the rear region 134 of the diaper 130, and are located inboard each longitudinal extending side edge 146. The fasteners 167 may be configured to encircle the hips of the wearer and engage the backsheet 138 of the front region 132 of the diaper 130 for holding the diaper on the wearer. Suitable fasteners are well known to those of skill in the art and can comprise adhesive tape tab fasteners, hook and loop fasteners, mushroom fasteners, snaps, pin, belts and the like, and combinations thereof. Desirably, the fasteners 167 are releasably engageable directly with the garment-facing surface of the backsheet 138. Desirably, the fasteners 167 comprise a mechanical fastening system.

By way of illustration only, various materials and methods for constructing other diapers are disclosed in PCT Patent Application WO 00/37009 published Jun. 29, 2000 by A. Fletcher et al.; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference to the extent they are consistent (i.e., not in conflict) herewith.

Having described the invention in rather full detail, it will be readily apparent to a person of ordinary skill that various changes and modifications may be made without departing from the spirit of the invention. All of such changes and modifications are contemplated as being within the scope of the invention, as defined by the subjoined claims.

1. A disposable assembly relating to the changing of a disposable garment, the assembly comprising:
a drape defined by a sheet having a terminal end opposite a free end and a drape width;
a package having a first compartment, a package width, and an accessible outer surface located opposite an attachment outer surface, the attachment outer surface defining an attachment area;
wherein the disposable garment is disposed in the first compartment; and
wherein the terminal end of the drape is attached to the attachment outer surface of the package so that only a portion of the attachment area is covered by the drape.

2. The disposable assembly of claim 1 wherein the drape is attached to the attachment outer surface of the package with a layer of adhesive.

3. The disposable assembly of claim 1 wherein the package further comprises a second compartment that is separate from the first compartment, wherein the second compartment contains an article.

4. The disposable assembly of claim 3 wherein the article comprises at least one wet wipe.

5. The disposable assembly of claim 3 wherein the accessible outer surface has a first opening thereon corresponding to the first compartment, and a second opening thereon corresponding to the second compartment.

6. The disposable assembly of claim 5 wherein the first opening or the second opening is covered by a removable panel.

7. The disposable assembly of claim 6 wherein the removable panel comprises a resealable adhesive, enabling the removable panel to be selectively removed and replaced from the accessible surface of the package.

8. The disposable assembly of claim 7 wherein the removable panel comprises indicia.

9. The disposable assembly of claim 3 wherein the accessible surface further comprises a frangible seal corresponding to the first compartment or the second compartment, wherein the frangible seal may be broken to allow selective entry into the first or second compartment.

10. The disposable assembly of claim 1 wherein the drape comprises a longitudinal fold and a lateral fold.

11. The disposable assembly of claim 10 wherein the drape longitudinal fold is substantially aligned with a package longitudinal fold.

12. The disposable assembly of claim 1 wherein the package comprises a longitudinal fold, and a closure member for maintaining the package in a folded storage condition.

13. The disposable assembly of claim 2 wherein the drape width is less than the package width.

14. A disposable assembly used to protect a surface for performing a task, the disposable assembly comprising:
a drape defined by a sheet having a terminal end opposite a free end;
a package having a first compartment and a second compartment, and an accessible outer surface opposite an attachment outer surface, the attachment outer surface defining an attachment area; and
a first article disposed in the first compartment and a second article disposed in the second compartment;
wherein the terminal end of the drape is attached to the attachment outer surface of the package so that only a portion of the attachment area is covered by the drape.

15. The disposable assembly of claim 14 wherein the first article and the second article are different types of articles.

16. The disposable assembly of claim 1 wherein the package is releasably attached to the drape.

17. A method of protecting a surface for performing a change of an absorbent garment comprising:
providing a disposable assembly in a storage condition, the disposable assembly comprising a drape defined by a sheet having a terminal end opposite a free end;
a package having a first compartment; and a disposable garment disposed in the first compartment; wherein the terminal end of the drape is attached to the attachment outer surface of the package so that only a portion of the attachment area is covered by the drape;
deploying the disposable assembly onto a support surface;
opening the first compartment; and
removing the absorbent garment.

18. The method of claim 17 wherein the deploying step further comprises the steps of:
opening the closure;
unfolding the package;
unfolding the drape; and
placing the support contact surface of the drape onto a support surface.

19. The method of claim 16 wherein the package is opened by removing a removable panel.

20. The method of claim 16 wherein the package is opened by breaking a frangible seal.
